(19) United States
(12) Patent Application Publication
(10) Pub. No.: US 2008/0268037 A1
(43) Pub. Date: Oct. 30, 2008

(54) Autonomous method for oral delivery of a healing substance to a target place in gastrointestinal tract of humans or animals

(76) Inventor: Igor Igorevich Stukanov, Toronto (CA)

Correspondence Address: IGOR STUKANOV, SUITE 405, 66 OAKMOUNT ROAD, TORONTO, ON, M6P 2M8, CA

(21) Appl. No.: 11/789,108

(22) Filed: Apr. 24, 2007

(51) Int. Cl. A61K 9/48; A61H 1/00; A61M 31/00; A61N 5/06

(52) U.S. Cl. 424/452; 601/2; 604/516; 607/88

(57) ABSTRACT

A method for oral delivery of new drugs, big molecules, proteins, and healing substances to a target place in the body, via digestive tract, using mobile self-controlled capsules with sensors.

### CROSS-REFERENCE TO RELATED APPLICATIONS

This application is related to provisional patent application U.S. 60/776,908 filed Feb. 24, 2006 and non provisional patent application U.S. Ser. No. 11/650,556 filed in 2007.

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

1. Field of the Invention

The current invention relates to oral autonomous high precision drug and healing substances/agents delivery methods in humans and animals.

2. Background Information

The field of advanced drug delivery is more than thirty years old. There are two main problems with oral delivering of healing substances such as proteins and biotech bio-molecules. The first problem is in the destruction of big healing substances such as proteins, peptides or big molecules by the agents in the digestive systems if these healing substances were taken orally. The second problem is in inaccuracy of targeting damaged regions. For example, chemotherapy may damage non-cancerous cells, because there is no way to detect with high precision cancerous cells inside the gastrointestinal tract by current chemical therapy and radiological therapy methods.

More than thousands of methods were proposed for solutions of these problems, which include the usage of electric field, and/or magnetic field, and/or ultrasound, and/or light, and/or enzymes, and/or viruses, and/or temperature, and/or pH, and/or mico-sized valves/chambers/pumps, and/or smart-microchips, and/or biopolymers, and/or nanoparticles, etc. Although some of these delivery technologies may be effective they are not cost efficient today and usually are restricted to the specific drugs, which can be delivered by these methods.

In the previous patent applications U.S. Ser. No. 11/650,556 the inventor suggests a reliable, low-cost, non-restricted to particular drugs, and convenient method for oral delivery of new drugs, big molecules, proteins, and other healing substances to the targeted place in the body via digestive tract, using currently available technologies of mobile wireless controlled capsules with sensors. The main disadvantage of the invention is non-autonomous control system, which rely on external expert/operator.

The current patent application describes a method for oral delivery of new drugs, molecules, proteins, and other healing substances to the targeted place in the body via digestive tract, using the autonomous mobile self-controlled capsules directed by control systems on the internal chip inside the capsule.

### BRIEF SUMMARY OF THE INVENTION

The current invention consists in using autonomous self-controlled capsules with imaging sensors to deliver healing substances to the target place in the gastro-digestive tract. Autonomous self-controlled capsules with imaging sensors and control system on the internal chip allow delivering of healing substances to a targeted region with very high precision, which allow to solve the problem of high precision targeting of damaged regions and to avoid exposure of non-damaged regions to healing substances. The second advantage of the current invention consist in delivery of peptides, proteins, and other big molecular structures, which are normally decomposed in the digestive tract if taken orally, into blood stream without their exposure to digestive machinery and without modifications in their structure and function. The third advantage of the current invention is autonomous self-control system on the internal chip, which allow using such pills in home environment without a need for specialized equipment and expert.

### BRIEF DESCRIPTION OF THE SEVERAL VIEWS OF THE DRAWINGS

Not applicable

### DETAILED DESCRIPTION OF THE INVENTION

The present invention is directed to a method for oral delivery of new drugs, big molecules, proteins, healing substances to a target place in the body, via digestive tract, using autonomous self-controlled mobile capsules with sensors.

The mobile capsules, for example modifications of PillCam manufactured by Given Imaging, Inc. or SmartPills manufactured by SmartPill Corporation have imaging sensors, chip on which targeted region image and control system are stored and healing substance delivery mechanism for delivery of healing substances with high precision, convenience for patients, and with low costs.

The internal control system on the chip compare images receiving from imaging sensors with the image of a target region, which is stored on the current or a separate chip. If the current image is similar to the pre-stored on the chip image of the target region then the control system activates the healing substance delivery mechanism for delivery of healing substance. This mechanism directs the healing substance to the targeted place.

Autonomous self-controlled mobile capsules with imaging sensors allow delivering healing substances to a targeted region with very high precision, which allow solving the problem of high precision targeting of damaged regions and avoiding exposure of non-damaged regions to healing substances.

The invention is described below on several examples.

### EXAMPLE 1

### Cancer in Gastrointestinal Tract

The delivery part of autonomous mobile self-controlled capsule is loaded with a healing substance, for example, molecules with special receptors with attached chemical or radioactive agent to treat cancerous cells. On the chip of the capsule an image of a targeted region of a cancer place is loaded. A patient swallows the capsule, which sends images of gastrointestinal tract to the control system as it travels in the tract. The control system analyzes the images captured by the imaging sensors of the capsule. When the control system detects that the capsule is close enough to the target region, the control system direct the healing substance to the target. This command is executed by capsule healing substance delivery mechanism, which may be any standard mechanism used today, for example, injectors, micro guns, pushers, pumps, etc. The healing substance directed to the target region, sticks to the target, and treats the cancer.

### EXAMPLE 2

### Delivering Proteins or Peptides Into the Blood System

The delivery part of mobile autonomous self-controlled capsule is loaded with proteins or peptides. On the chip of the capsule an image of a targeted region is loaded. A patient swallows the capsule, which sends images, captured by the imaging sensors, of gastrointestinal tract to the control system as it travels in the tract. The control system analyzes the images captured by the imaging sensors of the capsule. When the control system detects that the capsule passed the region where the proteins or peptides are destroyed by digestive enzymes in the gastrointestinal tract, the signal is sent to the healing substance delivery mechanism to release the proteins or peptides. This command is executed by capsule healing substance delivery mechanism, which release the proteins or peptides. These proteins or peptides then flow undamaged into the blood system.

The advantage of the current invention consist in delivery of peptides, proteins, and other big molecular structures, which are normally decomposed in digestive tract if taken orally, into blood stream without their exposure to digestive machinery and without modifications in their structure and function.

The next advantage of the current invention is the high accuracy of targeting the damaged regions.

The next advantage of the current invention is high degree of safety for using this method.

The next advantage of the current invention is possibility to use it without a need for specialized equipment and expert in home environment.

1. A method for delivery of a healing substance to a target place in gastrointestinal tract or blood system, comprising the following steps:
a). A healing substance is placed into a mobile self-controlled capsule with sensors and internal chip (or chips).
b). An image of a targeted region is loaded into memory of the chip
c). The capsule enters into the gastrointestinal tract via mouth of a patient with food or water and moves toward the targeted place.
d). The capsule is directed to the targeted place via control signals sent from a control unit in the internal chip. The signals from the sensors are sent to the control system on the internal chip inside the capsule.
e). The control system compares images received from the sensors with the image of the targeted place stored on the chip.
g). When the capsule reach the targeted place the control unit send signal to release the healing substance and this healing substance is released from the capsule and directed to the targeted place.

2. The method as in claim 1, where the healing substance is a composition of chemical elements in solid or fluid or gases form.

3. The method as in claim 1, where the healing substance is a set of electromagnetic waves.

4. The method as in claim 1, where the healing substance is a set of mechanical waves.

5. The method as in claim 1, where the healing substance is a set of sound or ultrasound waves.

6. The method as in claim 1, where the healing substance is a flow of physical particles.
